(19) United States
(12) Patent Application Publication
(10) Pub. No.: US 2008/0097451 A1
(43) Pub. Date: Apr. 24, 2008

(54) Surgical tool assembly for total knee arthroplasty

(75) Inventors: Ing-Ho Chen, Hua Lian City (TW); Po-Wen Chung, Kao Hsiung City (TW); Shou-Heng Pao, Chu Pei City (TW)

Correspondence Address: RABIN & Berdo, PC, 1101 14TH STREET, NW, SUITE 500, WASHINGTON, DC, 20005, US

(73) Assignees: United Orthopedic Corporation, Hsinchu (TW); Ing-Ho Chen, Hua Lian (TW)

(21) Appl. No.: 11/523,656

(22) Filed: Sep. 20, 2006

(51) Int. Cl. A61B 17/58; A61B 17/00

(52) U.S. Cl. 606/88; 606/103; 606/60; 606/87

(57) ABSTRACT

A surgical tool assembly for Total Knee Arthroplasty (TKA) including a femoral intra-medullary (IM) rod, a femoral intra-medullary (IM) alignment guide, a distal femoral alignment guide, and a distal femoral cutting guide. After the femoral IM alignment guide is selected to fit the valgus angle and utilized with the femoral IM rod with respect to the distal femur, the distal femoral alignment guide is integrated to the complex. Then, the distal femoral cutting guide is slid to the distal femoral alignment guide in the orientation from distal to proximal through the sliding concave integrated with the sliding track.

### FIELD OF THE INVENTION

The present invention relates to a surgical tool assembly used in Total Knee Arthroplasty (TKA) procedure, and more particularly, to a structure of a surgical tool assembly which enables the distal femoral cutting guide to be slid to the distal femoral alignment guide in the orientation from distal to proximal.

### BACKGROUND OF THE INVENTION

U.S. Pat. No. 5,676,668 published on Oct. 14, 1997, titled “FEMORAL LOCATING DEVICE ASSEMBLY” disclosed a femoral locating device used in a knee replacement surgical procedure, comprising a femoral locating device comprising: a body having a condyle interfacing end; a pivoting alignment element coupled to said body to pivot about a pivot axis with respect to said body; a lumen extending through said pivoting alignment element and through said condyle interfacing end for receiving an intramedullary rod therethrough; and a locking portion coupled to said body, said locking portion releasably engageable with said pivoting alignment element in a plurality of angular orientations with respect to the pivot axis to lock said pivoting alignment element in one of said plurality of angular orientations.

As mentioned, shortcomings of the device used in the knee replacement surgical procedure includes that the orientation followed by the cutting block from the proximal to distal and the whole size of the device too bulky to be utilized. Consequently, in the surgical procedure, the femoral quadriceps damage caused by an incision made on the knee between 20 cm to 25 cm causes serious pain, further more, harmful to the quality of rehabilitation. The disadvantages not only bring the inconvenience in the surgical procedure but reduce patients' quality of life after surgery.

### SUMMARY OF THE INVENTION

The present invention provides a surgical tool assembly adopted in TKA procedure including a femoral intra-medullary (IM) rod, a femoral intra-medullary (IM) alignment guide, a distal femoral alignment guide, and a distal femoral cutting guide. The feature of the invention provides a device that enables the distal femoral cutting guide to be slid to the distal femoral alignment guide in the orientation from distal to proximal through the sliding concave integrated with the sliding track after the valgus angle is set. By means of the device, the size of the incision may be narrowed down to provide more convenience to the knee replacement surgical procedure and to minimize the femoral quadriceps damage to facilitate patients' rehabilitation after surgery.

### BRIEF DESCRIPTIONS OF THE DRAWINGS

FIG. 1 illustrates the exploded view of the present invention

FIGS. 2a-2b illustrate the vertical views of the femoral IM alignment guide with 5° valgus angle of horizontal openings;

FIGS. 2c-2d illustrate the vertical views of the femoral IM alignment guide with 7° valgus angle of horizontal openings;

FIGS. 2e-2f illustrate the vertical views of the femoral IM alignment guide with 9° valgus angle of horizontal openings;

FIGS. 3a-3b illustrate the vertical views and front views of the distal femoral cutting guide before the positioning device spun;

FIGS. 3c-3d illustrate the vertical views and front views of the distal femoral cutting guide after the positioning device spun;

FIG. 4 illustrates the perspective view of adopting the femoral IM rod and the femoral IM alignment guide;

FIG. 5 illustrates the perspective view of adopting the distal femoral alignment guide;

FIG. 6 illustrates the perspective view of adopting the distal femoral cutting guide;

FIG. 7 illustrates the perspective view of fixing the distal femoral cutting guide; and

FIG. 8 illustrates the perspective view of the final distal femoral cutting.

### DETAILED DESCRIPTIONS OF THE PREFERRED EMBODIMENT

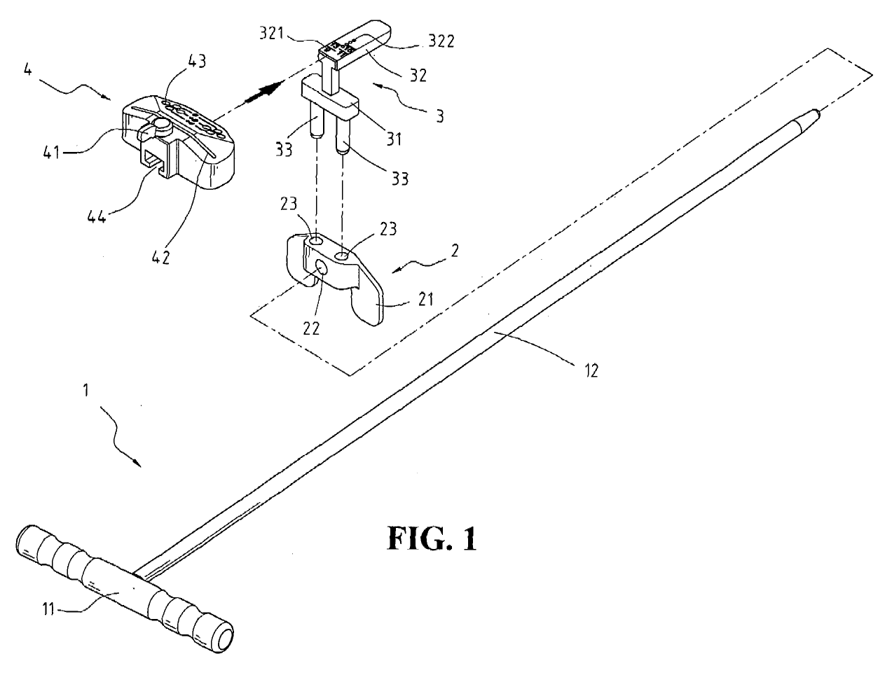

FIG. 1 illustrates the exploded view of surgical tool assembly adopted in TKA, including a femoral IM rod 1, a femoral IM alignment guide 2, a distal femoral alignment guide 3, and a distal femoral cutting guide 4. The femoral IM rod 1 is a T-shaped structure formed by a shaft 11 and a rod 12. The femoral IM alignment guide 2 comprises a wing section 21 at both sides respectively, a horizontal opening 22 for receiving the IM rod 1 in the middle and two vertical openings 23 on the top. The distal femoral alignment guide 3 includes a platform 31, a sliding track 32 on the platform 31, and two legs 33 beneath inserted into the two vertical openings 23. The distal femoral cutting guide 4 comprises a positioning device 41, a saw slot 42, fixing holes 43, and a sliding concave 44 for receiving the sliding track 31.

The structure of the invention utilizes the distal femoral cutting guide slid to the distal femoral alignment guide in the orientation from distal to proximal to reduce the size of the incision and provide more convenience to the knee replacement surgical procedure and to minimize the femoral quadriceps damage to facilitate patients' rehabilitation after surgery.

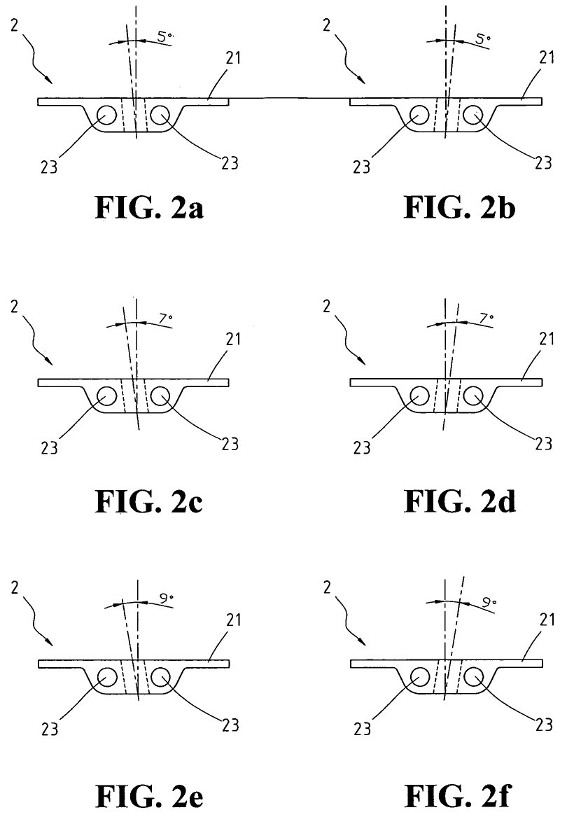

Referring to FIGS. 2a-2f, individually illustrating the vertical views of the femoral IM alignment guide 2 with 5°, 7° and 9° valgus angles. The angles coincide with the differences between the mechanical axes and the anatomical axes.

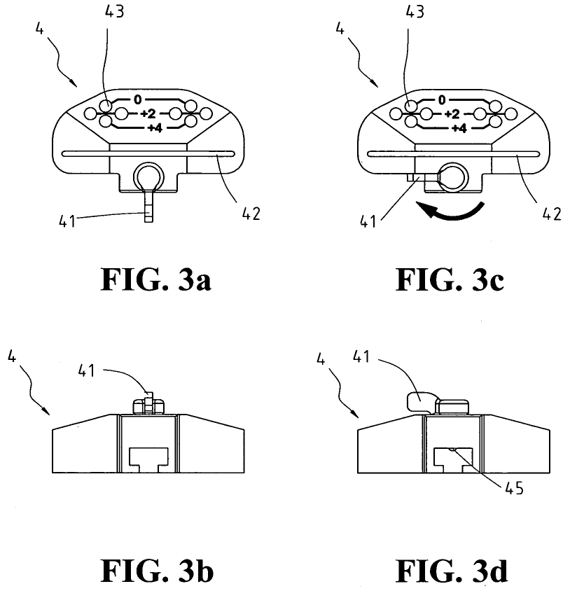

As illustrated in FIGS. 3a-3b, the positioning device protruding 45 hides inside the distal femoral cutting guide 4 before the positioning device 41 spun 41. As illustrated in FIGS. 3c-3d, the positioning device protruding 45 extended to fasten the distal femoral cutting guide 4 after the positioning device 41 spun.

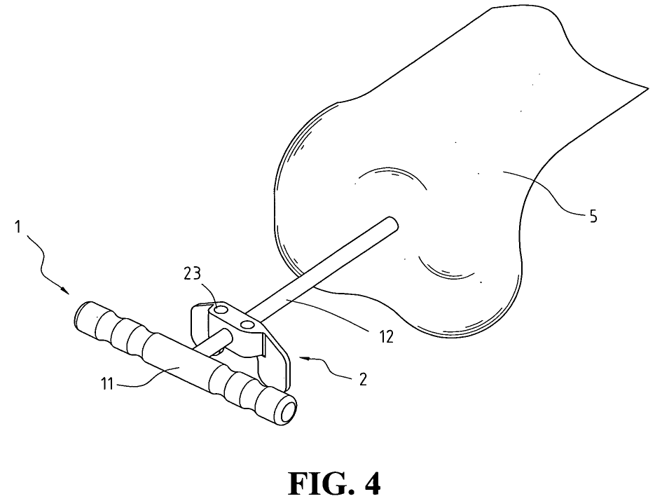

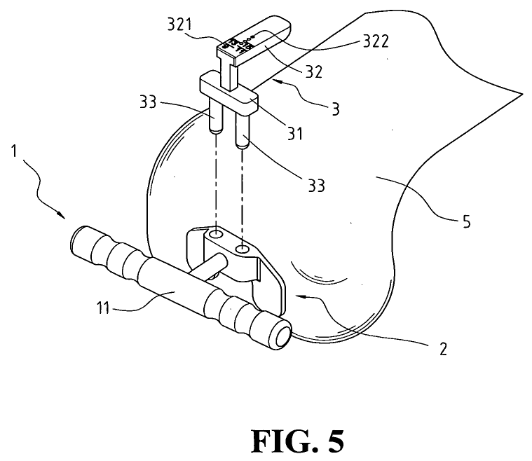

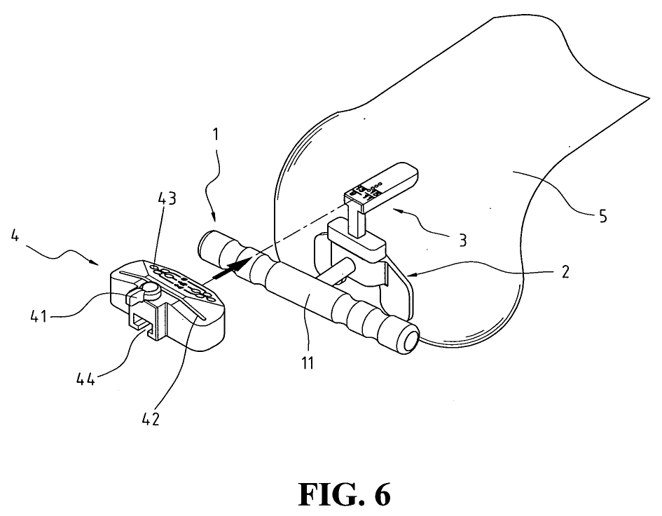

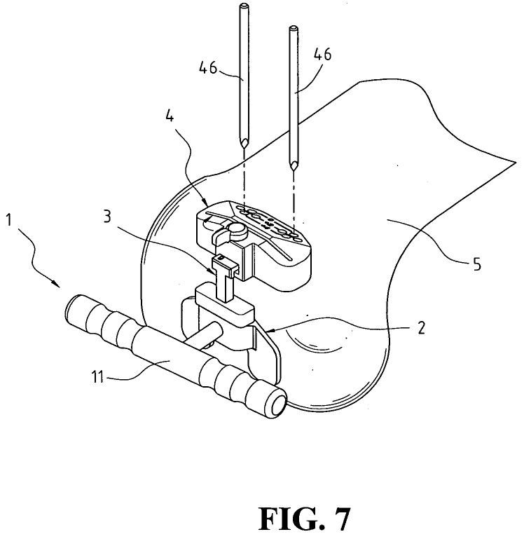

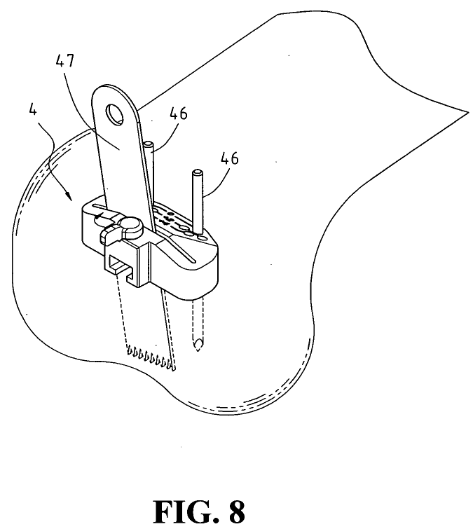

As illustrated in FIG. 4, the rod 12 is inserted throughout the horizontal opening 22 of the femoral IM alignment guide 2 into the femur 5 to locate the femoral IM alignment guide 2 with respect to the femur 5. The horizontal openings are with the valgus angles coinciding to the differences between the mechanical axes and the anatomical axes. As illustrated in FIG. 5, the distal femoral alignment guide 3 is combined with the femoral IM alignment guide 2 by inserting the two legs 33 into the corresponding vertical openings 23 and the distal femoral alignment guide 3 is adjusted to a proper height according to the thickness of the distal femur 5. Then referring to FIG. 6, in the orientation from distal to proximal, the distal femoral cutting guide 4 is slid to the distal femoral alignment guide 3 by integrating the sliding concave 44 with the sliding track 32 and the size of the distal resection depends on the distal resection numbers 321 carved on the sliding track 32. There are four locking concaves on the sliding track of the distal femoral alignment guide 3. After the positioning device 41 spun, the positioning device protruding 45 extended to locate in the proper locking concave 322 of the distal femoral alignment guide 3 to lock the distal femoral cutting guide 4. The pins 46 are drilled through the fixing holes 43 into the distal femur 5 for securing the distal femoral cutting guide 4 with respect to the medullary canal and distal condyle as illustrated in FIG. 7. The femoral IM rod 1, the femoral IM alignment guide 2 and the distal femoral alignment guide 3 may then be removed and the distal cut may be performed by putting the saw 47 into the saw slot 42 as illustrated in FIG. 8 to form a cutting plane perpendicular to the mechanical axis.

While the invention has been particularly shown and described with reference to the preferred embodiments thereof, these are, of course, merely examples to help clarify the invention and are not intended to limit the invention. It will be understood by those skilled in the art that various changes, modifications, and alterations in form and details may be made therein without departing from the spirit and scope of the invention, as set forth in the following claims.

What is claimed is:

1. A surgical tool assembly for Total Knee Arthroplasty (TKA) comprising:
a femoral intra-medullary (IM) rod, a T-shaped structure formed by a shaft and a rod;
a femoral intra-medullary (IM) alignment guide with a wing section at both sides respectively, a horizontal opening for receiving the IM rod in the middle, two vertical openings on the top;
a distal femoral alignment guide with a platform, a sliding track on the platform, two legs beneath inserted into the two vertical openings; and
a distal femoral cutting guide with a positioning device, a saw slot, fixing holes, and a sliding concave for receiving the sliding track;
the characteristic is the structure of the invention, enabling the distal femoral cutting guide slid to the distal femoral alignment guide in the orientation from distal to proximal to narrow down the size of the incision, thus providing more convenience to the knee replacement surgical procedure and minimizing the femoral quadriceps damage to facilitate patients' rehabilitation after surgery.

2. The surgical tool assembly of claim 1, wherein the horizontal openings are individually with different valgus angles coinciding to the differences between the mechanical axes and the anatomical axes.

3. The surgical tool assembly of claim 1, further including distal resection numbers carved on the sliding track to show the size of distal resection.

4. The surgical tool assembly of claim 1, further including locking concaves on the sliding track for the distal femoral cutting guide fastened on the distal femoral alignment guide.

5. The surgical tool assembly of claim 1, wherein the positioning device corresponding to the locking concave on the sliding track makes the distal femoral cutting guide fastened on the distal femoral alignment guide.

6. The surgical tool assembly of claim 1, wherein the saw slot guides the saw to make an initial cut to form a cutting plane perpendicular to the mechanical axis.

7. The surgical tool assembly of claim 1, wherein the fixing holes on the distal femoral cutting guide receive pins drilled into the femur to fix the distal femoral cutting guide with respect to the medullary canal and distal condyle.

8. The surgical tool assembly of claim 1, wherein the sliding concave is integrated with the sliding track to enable the distal femoral cutting guide slid to the distal femoral alignment guide in the orientation from distal to proximal.

9. The surgical tool assembly of claim 2, wherein the valgus angles of horizontal openings are between 0 degree and 10 degree.

10. The surgical tool assembly of claim 4, wherein there are 4 locking concaves on the sliding track.
